(19) United States
(12) Patent Application Publication
(10) Pub. No.: US 2001/0036484 A1
(43) Pub. Date: Nov. 1, 2001

(54) Composition comprising ingredients of panax ginseng and ginko biloba

(76) Inventor: Fabio Soldati, Savosa (CH)

Correspondence Address: BOEHRINGER INGELHEIM CORPORATION, 900 RIDGEBURY ROAD, P O BOX 368, RIDGEFIELD, CT, 06877, US

(21) Appl. No.: 09/740,665

(22) Filed: Dec. 18, 2000

Related U.S. Application Data
(60) Provisional application No. 60/172,501, filed on Dec. 17, 1999.

(51) Int. Cl. A61K 35/78

(52) U.S. Cl. 424/728; 424/752

(57) ABSTRACT

A method of improving blood flow circulation comprising the administration of the combination of the medicinal plants Panax ginseng and Ginkgo biloba or the combination of extracts of Panax ginseng and Ginkgo biloba.

Said combination can be used for the treatment or the prevention of pathologies related to impaired blood flow circulation or to impaired blood flow velocity, particularly of cardio- and cerebrovascular diseases.

### RELATED APPLICATION

The benefit of prior provisional application Ser. No. 60/172,501 filed Dec. 17, 1999 is hereby claimed.

### FIELD OF THE INVENTION

The present invention relates to a novel use of a combination of the medicinal plants Panax ginseng and Ginkgo biloba or the combination of extracts of both plants for improving hemorrheological and circulatory characteristics of blood, to the use of said combination for the manufacture of a medicament for treating or preventing pathologies related to impaired blood flow circulation and to a method of treatment or prevention of pathologies related to an impaired blood flow circulation.

### BACKGROUND TO THE INVENTION

The Panax ginseng root and its extracts (PG) have been recognised as an effective tonic or roborant of health for thousands of years in China. The Ginkgo biloba leaves and its extracts (GB) were also introduced as medicine in the treatment of many diseases throughout a long history in China.

Only during the last decades were the efficacy of PG and GB as treatment of many pathological processes proven scientifically. PG showed a significant action to stimulate the immune condition to improve pulmonary function and to increase oxygen uptake. Further, it was proven that PG is protective against experimental damages provoked by free radicals in different organs.

It is also believed that ginkgolides have a potential effect on immunological and inflammatory disorders. Increasing attention has been paid to the specific PAF (platelet-activating factor) receptor antagonist effects of several compounds isolated from Ginkgo biloba. Intensive research is being carried out for exploring the influence of Ginkgo biloba on circulatory dysfunctions, such as ischemic states in the brain, heart, gastrointestinal tract and other organs.

### SUMMARY OF THE INVENTION

It has surprisingly been found that the administration of the combination of the medicinal plants Panax ginseng and Ginkgo biloba or the combination of extracts of Panax ginseng and Ginkgo biloba significantly increases in a synergistic way blood flow and blood velocity. These effects, found during experimental pharmacological tests, were unexpected as neither Panax ginseng nor Ginkgo biloba alone show such effects.

It is a primary object of the present invention to provide a medication and/or a dietary supplement to improve the blood flow circulation and blood flow velocity of warm blooded animals and humans.

It is a further object of the present invention to provide a medication and/or a dietary supplement to improve the blood flow circulation and blood flow velocity of warm blooded animals and humans by administering formulations comprising herbal ingredients, wherein the medication and/or dietary supplement is manufactured pursuant to a controlled process that preserves the herbal curing qualities of the ingredients.

It is still a further object of the present invention to provide a medication and/or a dietary supplement for improving the blood flow circulation and blood flow velocity of warm blooded animals and humans comprising herbal ingredients having minimal or no side effects and thus being safe for internal consumption.

It is a further object of this invention to provide a method for improving blood flow circulation and blood flow velocity of warm blooded animals and humans.

It is a further object of the present invention to provide a method for the treatment of or prophylaxis of pathologies of warm blooded animals and humans related to an impaired blood flow circulation or an impaired blood flow velocity of the cardiovascular system or the cerebrovascular system.

### BRIEF DESCRIPTION OF FIGURES

FIG. 1 shows the system for simultaneous incubation, cultivation and intravital TV-microscopic observation on microcirculation of chick embryo chorioallantoic membrane.

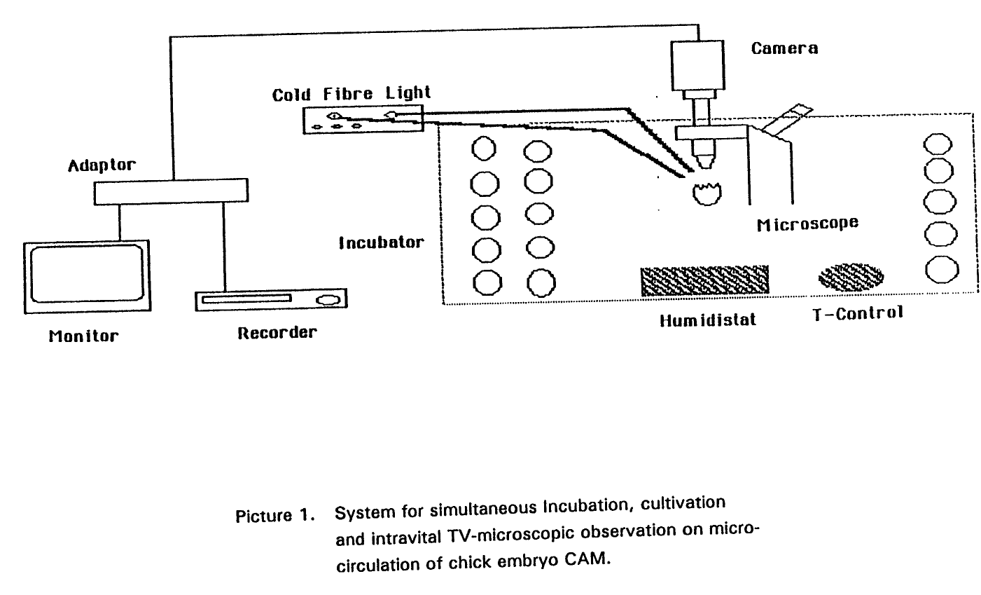

FIG. 2 shows a computer image analysis of vascular hemodynamic

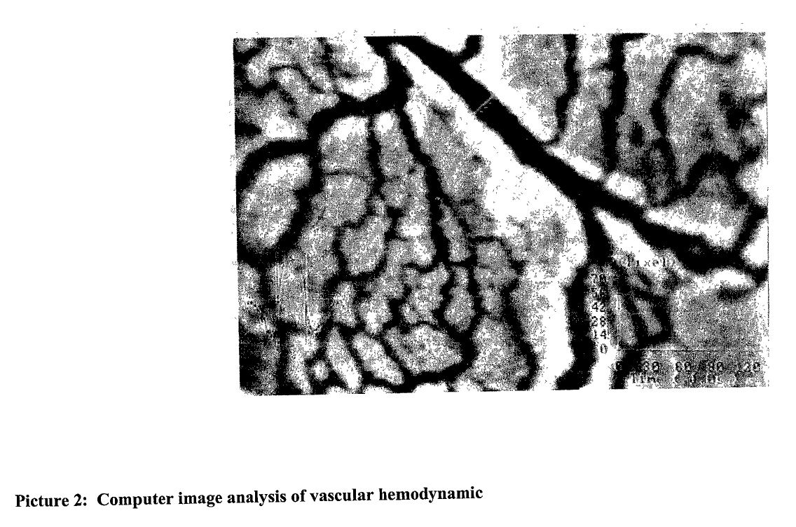

FIG. 3 shows the effect on blood flow velocity of control (physiological solution), of the standardised Ginkgo biloba extract (GK501), of the standardised Panax ginseng extract (G115) and of the combination of both extracts (PHL-00701).

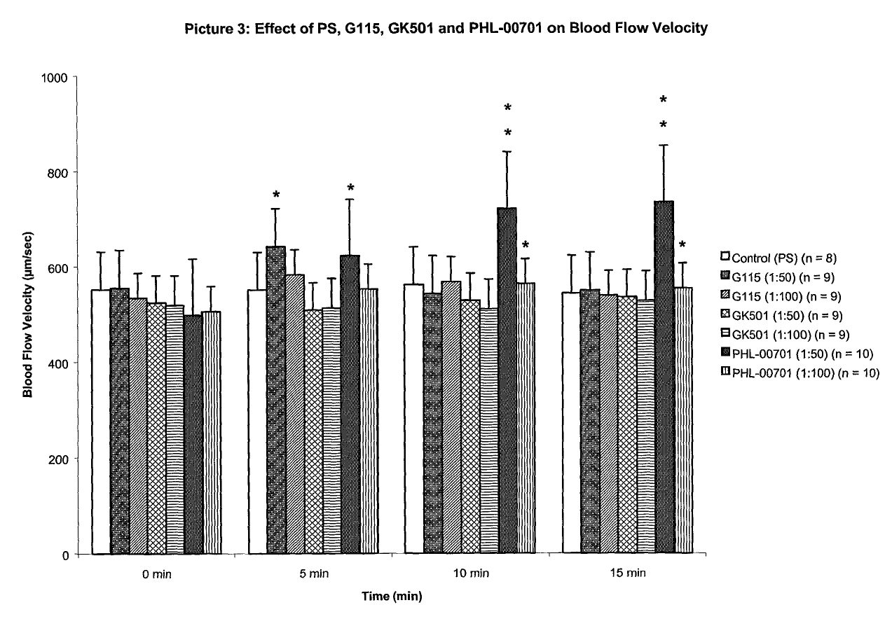

### DETAILED DESCRIPTION OF THE INVENTION

The invention relates to a method to improve blood flow circulation and blood flow velocity of warm blooded animals and humans by co-administration of or a combination-administration of:

(i) the medicinal plant Panax ginseng, extracts thereof and/or the principle active substances thereof; and

(ii) the medicinal plant Ginkgo biloba, extracts thereof and/or the principle active substances thereof.

In this context under the term “co-administration” are meant that each of the two components (i) and (ii) as described above are administered separately but within a close timely relationship. The two components (i) and (ii) are taken within a break of about 12 hours or less. Preferably, both of them are taken within 4 hours, more preferably within one hour and most preferably together.

Under the term “combination-administration” is meant that synergistically effective amounts of both components (i) and (ii) are present in one formulation.

The two components (i) and (ii) may be formulated independently of each other or together in one formulation. For example, the formulation comprises dried Ginseng roots or Ginkgo leaves or other plant components, that optionally are powdered, the formulation may be in the form of tablets, coated tablets, pulvers, pulvers in capsules, syrups, solutions or suspensions, for example, on the basis of water, ethanol or a mixture thereof, dragees, gels, injections or any other suitable manner well known to the skilled person. Preferred are oral administration forms.

Under the term “plant” is understood the plant itself as well as plant components comprising the active ingredients, for example, the leaves or roots as mentioned above. Preferably, the plant or plant components are dried. Optionally, they may be powdered.

Under the term “extracts” is meant that the plants or plant components are extracted with a suitable solvent like water, ethanol a mixture thereof, oils or any other suitable solvent well known in the state of the art of extracting plants. These extracts can be used as such if pharmacologically acceptable or the solvent of the resulting solutions is removed and the residue is used as such or after further work up, for example, after resolving or resuspending in a pharmacologically suitable solvent.

Under the term “principle active ingredients” is meant all active ingredients that are mainly responsible for the pharmacological effect. Preferably, the formulation comprises all those ingredients of the plant of interest that are responsible for at least 75 per cent, more preferably at least 90 percent of the pharmacological effect. These active ingredients may be won from the plants or synthesised chemically.

This method can be used for the treatment of pathologies or prevention of pathologies related to impaired blood flow circulation or impaired blood flow velocity of the cardiovascular system or the cerebrovascular system.

The invention further relates to the use of a combination of:

(i) the plant Panax ginseng, extracts thereof and/or the principle active substances thereof; and

(ii) the plant Ginkgo biloba, extracts thereof and/or the principle active substances thereof

for the manufacture of a medicament for the treatment of pathologies or prevention of pathologies related to impaired blood flow circulation or impaired blood flow velocity of the cardiovascular system or the cerebrovascular system.

Under the term “pathologies related to” is meant any disease that is caused or amplified by impaired blood flow circulation or impaired blood flow velocity of the cardiovascular system or the cerebrovascular system as well as diseases that cause or amplify such an impaired blood flow velocity of the cardiovascular system or the cerebrovascular system. In this context it is of no importance if the impaired blood flow velocity is a primary or secondary effect of the disease.

The invention further relates to a dietary supplement comprising:

(iii) the plant Panax ginseng, extracts thereof and/or the principle active substances thereof; and

(iv) the plant Ginkgo biloba, extracts thereof and/or the principle active substances thereof.

Under the term “dietary supplement” is meant that the components (i) and (ii) may be used without prescription by a third party, for example, a physician. The components may be taken together with meals or separated thereof, on a daily basis or only sometimes. The components may be formulated as described above, for example.

### EXAMPLES

A preferred embodiment concerns a method according to the above described in that Ginseng extract is used containing among other substances ginsenosides and polysaccharides, preferably containing at least 3%, more preferably 3.5 to 5.0%, most preferably 3.6 to 4.4% ginsenosides. Ginkgo extract is used containing among other substances ginkgo flavone glycosides and terpene lactones, preferably containing at least 20%, more preferably 21.0 to 30%, most preferably 22.0 to 27.0% ginkgo flavone glycosides and 2 to 10, preferably 4 to 8, more preferably 5.0 to 7.0%, most preferably about 6% terpene lactones.

Preferably, the method comprises administration of 300 to 1000 mg, more preferably 300 to 990 mg, most preferably 320, 640 or 960 mg of a composition comprising 100 mg Panax ginseng extract, preferably Ginseng extract G115 and 60 mg Ginkgo biloba extract, preferably Ginkgo extract GK501, which is commercially available from Pharmaton S. A., Switzerland as PHL-00701 or under the tradename GINCOSAN ®.

Experimental Preparation:

The chick chorioallantoic membrane (CAM) has been used as a test preparation during recent years. CAM has been established in many laboratories as an optimal model for screening substances that show circulatory activities. In these studies, the common and usual way was to culture the chicken embryos in petri dishes or plastic bags without their egg shell, and the investigations were performed in vitro. During these experiments, the embryo had to be removed from the incubator, and the microscopic observation had to be done at room temperature. Many reports proved, that by using this shell-less culture technique most of the embryos died 2-4 days before hatching, because the vital function of the egg shell could not be replaced by plastic or glass. This model has been improved and the microvascular activity of the CAM can be studied inside of the incubator during natural embryonic development in vivo in the fertile eggshell without glass or plastic (Xiu, R. J. et al., Proc. Vth World Congress Microcirc. USA, 1991).

Resource of Fertilised Eggs:

The high quality standard fertilised eggs were used each session of the experiments. Before cultivation, the eggs were randomly divided into 4 groups:

(1) Control (Physiological solution, P.S.),

(2) G115;

(3) GK501;

(4) PHL-00701

Resource of Substances:

1. G115: Standardised Panax ginseng extract G115, (Pharmaton S. A., Lugano, Switzerland)

2. GK501: Standardised Ginkgo biloba extract GK501 (Pharnaton S. A., Lugano, Switzerland)

3. PHL-00701: A combination of G115 and GK501 in the ratio of 5:3 (Pharmaton S. A., Lugano, Switzerland)

Preparation of the Stock Solutions:

The G115 stock solution, with a concentration of 10 mg/ml, was prepared in ethanol 40%. The GK stock solution, with a concentration of 10 mg/ml, was prepared in ethanol 94%. After solubilization, the insoluble part was removed by filtration through a standard 0.4 μm filter. Then, the PHL-00701 stock solution was prepared by mixing the G115 and GK501 stock solutions in a ratio of 5:3.

The stock solutions were diluted to 1:50 and 1:100 using physiological solution (P.S.). For each experiment, the solutions were freshly prepared and placed into the egg-incubator (37° C.) for 40 minutes before use. As a control, the P.S. with ethanol in the same concentration as in the tested solutions was used.

Equipments and Techniques:

An infant incubator was used for simultaneous cultivation of the chick embryo and circulatory observation on the CAM. Forty to sixty fertile eggs could be put into the incubator (temperature: 37° C., humidity: 60%) at one session. On day 8, a window of 2×2 cm was opened on the air sac end of the egg for circulatory observation. The operation was done under sterile conditions in the same incubator.

The circulation of the exposed CAM was observed by a long focus stereo microscope fixed inside the incubator. A cold fibre light was used for illumination. The vascular image processed via a high resolution colour TV-camera and colour monitor was recorded and then the parameters of circulation were measured and analysed via a computer-assisted processing system (Picture 1).

Circulation of CAM and its Observation:

At the beginning of the experiment, the circulatory activities of a focused area of each CAM were recorded for 5 minutes, as an equilibrating period and a self-control condition. The substances to be examined were then dropped on the surface of the CAM very carefully via micropipet. The behaviour of the vasculature was recorded continuously for 15 minutes after administration of each substance.

Computer Image Processing System:

A microcirculatory image processing system was used to improve the quality of vascular images and to extract more information from the processed microcirculatory image. A dynamic and static image separation processing mode was used in which one or more frames of interest can be separated out while the dynamic image is being analysed frame by frame. The microvascular contour line, diameter, blood flow velocity and other parameters can be automatically extracted from any area of network and can be measured (Picture 2), (see R. J. Xiu and X. Y. Ying; A non-invasive synchronous method for measurement of macro- and microcirculatory parameters in clinical research; Proc. of Chinese Academy of Medical Sciences and PUMC, Vol. 2, No. 2, 1987; X. Y. Ying and R. J. Xiu; Feature Extraction of Digital Microvessel images for Microvascular Network Analysis; Proc. V^{th} World Congress for Microcirculation, USA, 1991).

Statistical Analysis:

Mean value and Standard Deviation (SD) were used to analyse the variance of each group of data. The classical student's T-test for paired data was used to determine statistical significance for comparison of the data measured at different time points (0, 5, 10, 15 min) from the same group of vessels. P values of 0.05 or less were considered to be significant.

Results:

Table 1 and Picture 3 demonstrate the effect of P.S., G115, GK501 and PHL-00701 on blood flow velocity in vessels.

Table 1: Effect of P.S., G115, GK501 and PHL-00701 on Blood Flow Velocity

| | Number | Blood Flow velocity (μm/sec) | | | |
|---|---|---|---|---|---|
| | of | at time of measurements | | | |
| Group | vessels | 0 min | 5 min | 10 min | 15 min |
| Control | 8 | 552.0 ± 81.3 | 551.0 ± 80.2 | 562.0 ± 76.9 | 544.0 ± 77.9 |
| G115 (1:50) | 9 | 555.0 ± 63.2 | *642.0 ± 103.0 | 543.0 ± 94.2 | 550.0 ± 58.4 |
| G115 (1:100) | 9 | 534.0 ± 55.9 | 583.0 ± 56.4 | 568.0 ± 51.8 | 539.0 ± 45.8 |
| GK501 (1:50) | 9 | 524.0 ± 52.6 | 509.0 ± 68.6 | 529.0 ± 57.3 | 536.0 ± 49.6 |
| GK501 (1:100) | 9 | 519.0 ± 57.5 | 513.0 ± 74.0 | 511.0 ± 66.0 | 528.0 ± 51.0 |
| PHL-00701 (1:50) | 10 | 498.0 ± 71.7 | *623.0 ± 126.0 | **722.0 ± 142.0 | **735.0 ± 133.0 |
| PHL-00701 (1:100) | 10 | 506.0 ± 57.7 | 553.0 ± 60.6 | *564.0 ± 47.3 | *554.0 ± 43.9 |

Mean value ± SD:
*P < 0.05,
**P < 0.01

Each vessel was selected from one CAM of one individual fertilised egg.

The statistical analysis was done against the individual control (0 min) of each measurement.

Effect of Control (Physiological Solution):

The blood flow velocity was in a range of 552±81.3/551±80.2 and 562±76.9/544±77.9 (μm/sec), respectively.

Effect of G115:

Under G115 administration, the blood flow velocity was increased only after 5 min of the application and for the high dose, at all other times after application and for lower doses, there was not a significant increase of the blood flow velocity.

Effect of GK501:

In the low and high concentrations, the GK501 showed no statistically significant effect on the blood flow velocity.

Effect of PHL-00701:

The combination of both extracts G115 and GK501 caused a very significant increase of blood flow velocity which was not achieved with the application of the single extracts.

The significant high speed of blood flow appeared within the first 5 min after application of PHL-00701 and the blood flow velocity was very significantly increased after 10 and 15 min of measurement. This surprising result is due to the synergistic activity when the extracts of Panax ginseng and Ginkgo biloba are combined. The extent of the blood flow velocity reached with PHL-00701 cannot be reached with the application of the extracts of the single plants of Panax ginseng or Ginkgo biloba. These results confirm that the combination of Panax ginseng and Ginkgo biloba improves the circulatory blood perfusion.

This surprising discovery can be implemented for the treatment or prevention of pathologies related to impaired blood flow circulation as well as with cardio- and cerebrovascular diseases.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All publications and patent applications cited herein are incorporated by reference in their entireties.

What is claimed is:

1. A method to improve the blood flow circulation and blood flow velocity of warm blooded animals and humans by co-administration of or a combination-administration of:
(i) the medicinal plant Panax ginseng, extracts thereof and/or the principle active substances thereof; and
(ii) the medicinal plant Ginkgo biloba, extracts thereof and/or the principle active substances thereof.

2. A method according to claim 1 for the treatment of pathologies related to an impaired blood flow circulation or an impaired blood flow velocity of the cardiovascular system.

3. A method according to claim 1 for the treatment of pathologies related to an impaired blood flow circulation or an impaired blood flow velocity of the cerebrovascular system.

4. A method according to claim 1 for the prevention of pathologies related to an impaired blood flow circulation or an impaired blood flow velocity of the cardiovascular system.

5. A method according to claim 1 for the prevention of pathologies related to an impaired blood flow circulation or an impaired blood flow velocity of the cerebrovascular system.

6. Dietary supplement comprising:
(i) the plant Panax ginseng, extracts thereof and/or the principle active substances thereof; and
(ii) the plant Ginkgo biloba, extracts thereof and/or the principle active substances thereof.
